(19) United States
(12) Patent Application Publication
(10) Pub. No.: US 2017/0319289 A1
(43) Pub. Date: Nov. 9, 2017

(54) SYSTEM FOR ROBOT-ASSISTED MEDICAL TREATMENT

(71) Applicant: KUKA Roboter GmbH, Augsburg (DE)

(72) Inventor: Thomas NEFF, München (DE)

(21) Appl. No.: 15/534,758

(22) PCT Filed: Nov. 26, 2015
(86) PCT No.: PCT/EP2015/077779

§ 371 Date: Jun. 9, 2017

(30) Foreign Application Priority Data
Dec. 17, 2014 (DE) 10 2014 226 240.2

(51) Int. Cl. A61B 90/00; A61B 34/30; A61B 5/00; A61B 10/02; A61B 8/08; A61B 34/20

(52) U.S. Cl. CPC A61B 90/361; A61B 10/0233; A61B 34/30; A61B 5/0082; A61B 90/39; A61B 8/0841; A61B 2034/2063; A61B 2090/065; A61B 2034/2055; A61B 2090/3983; A61B 2090/3937

(57) ABSTRACT

A system (1) and a method for robot-assisted medical treatment of a patient. The system comprises a manipulator (20), a medical visualization device (30), which is mounted on the manipulator (20) in order to be moved by said manipulator; and a medical instrument (40), which is provided with at least one marker (41) in order that the location of the medical instrument (40) can be detected. A control device (10) moves the manipulator such that the visualization device is orientated depending on the location or position of the medical instrument.

### 1. TECHNICAL FIELD

The present invention relates to a system and a method for robot-assisted medical treatment of a patient.

### 2. TECHNICAL BACKGROUND

Using medical visualization devices, such as ultrasound devices, assisted medical examinations or treatments are today considered to be standard procedures in medicine. One example of such a medical treatment is a special biopsy, which is monitored with ultrasound in order to realize the removal of a tissue sample from lymph nodes in the neck using a fine needle for the purpose of cytological examination in the case of a suspected tumor (e.g. Hodgkin's lymphoma). In this procedure, the physician performing the procedure holds the biopsy needle in one hand and in the other hand the ultrasonic probe in order to monitor the arrival at the target region (e.g. suspected tumor) using an ultrasound image and so that, when approaching the target region, no damage occurs to structures that need to be protected, such as blood vessels, for example.

The problem here is that the displayable sonic plane is only a few millimeters thick. In order for the instrument to be visible in the ultrasonic plane, it must lie precisely within this plane. The important information, namely, the location and orientation of the needle tip relative to the target region, is relatively difficult to represent. This requires that the transducer head is moved in the correct position and orientation on the surface of the body. During surgery it is very difficult, in particular for inexperienced users, to hold the ultrasonic transducer head and the needle in such a way that the entire needle or at least specifically the tip of the needle is depicted.

Methods are known from the prior art in which the ultrasonic transducer head is guided by means of a manipulator, in particular a robot. For example, a robot system is known from document U.S. Pat. No. 7,753,851, in which a probe is mounted on the hand flange of the robot, and can be moved by the robot. Compared with manual operation of the probe, the robot-assisted treatment permits particularly precise orientation of the probe.

Document US 2004/0010190 A1 describes a robot with a medical visualization device (e.g. ultrasonic probe or ultrasonic transducer head). The objective of this application is the depiction of a structure of interest inside the body. The system allows the user (physician) to change the position of the device if it is in the way, and the robot controller then automatically adjusts the orientation of the device in such a way that the structure of interest is still depicted.

In addition, a robot-assisted ultrasound examination of a patient is known from document U.S. Pat. No. 6,425,865, in which the ultrasonic probe is mounted on a robot and the robot is manually controlled by the surgeon via a joystick or the like.

One disadvantage of some of the above methods is that, while the medical device is positioned with the aid of the robot, it is nevertheless still up to the user to realize the correct positioning. The robot-assisted methods, in which the robot assumes the task of reorienting the medical device when the user has pushed the device to the side for example, are not very flexible because the robot can still only target a previously defined point. As a general rule it is also a problem inherent in particular to ultrasonic applications that, even with the aid of the robot, it is not always easy for the user to correctly orientate the image plane in order to obtain the required image information. The reason for this is the thin sonic plane, which can change significantly even in the case of small movements of the transducer head on the surface of the body. Converting the image information into compensatory movement is relatively difficult for a person because a complex transfer step is required to achieve the eye-hand coordination.

The problem addressed by the present invention is therefore to provide an improved system and method for robot-assisted medical treatment of a patient which makes it possible to avoid or minimize the disadvantages of the prior art. A particular problem addressed by the present invention is to simplify the orientation of a medical visualization device, such as an ultrasonic probe for example, so as to make the surgeon's task easier.

These problems as well as others, which will emerge from the detailed description below, are solved by the subject matter of the independent claims 1 and 9.

### 3. CONTENT OF THE INVENTION

The invention relates to a system for robot-assisted medical treatment of a patient, said system comprising a manipulator, in particular a multiaxial articulated robot, and a medical visualization device, which is mounted on the manipulator in order to be moved by said manipulator. A medical instrument is also provided, which is provided with at least one marker in order that the location of the medical instrument can be detected, and also comprising a control device, which is configured to determine the location of the medical instrument with the aid of the marker and to move the manipulator with the medical visualization device depending on the determined location of the medical instrument. The medical instrument, e.g., a biopsy needle, a catheter, a radiation source, etc., is preferably directly manually guided by the surgeon, however, it can also be mounted on an additional manipulator and guided by means of this additional manipulator. The marker on the medical instrument is detected by a suitable sensor for example in order that the location of the marker in space can be detected and thus—because the offset of the marker and the instrument is known—the location of the instrument. The sensor is assigned to the control device, i.e., it is part of the control device, for example, so that the location of the instrument can be determined by the control device with the aid of the detected location of the marker. The term “marker” shall be understood in its broadest sense here and can, for example, also include the specified kinematics of a manipulator when the instrument is not guided manually, but rather with the aid of an additional manipulator. The only important thing is that the controller can determine the location of the instrument.

The controller moves the manipulator depending on the determined location of the instrument. The manipulator preferably follows a movement of the instrument in such a way that the visualization device always makes a desired area visible or such that a desired area can always be viewed by means of the visualization device. The medical visualization device itself is to be understood here simply as an element or device which supplies the data for visualization. This data is then sent to a data processor or computer and appropriately processed by this computer and displayed on a human-machine interface or a monitor, so that a treating physician can interpret/record it. This data transfer preferably occurs in a wireless or wired manner.

The manipulator is particularly preferably moved in such a way that the medical visualization device detects at least a part of the instrument, such as the tip of a biopsy needle, for example. When a transducer head is used, the optimal location for example of the head relative to the (biopsy) needle is fixed within a tolerance range. The tolerance range is determined by the spatial expansion of the (biopsy) needle and the sonic plane. The optimal position of the ultrasonic transducer head can be determined from this (relatively) fixed relationship between (biopsy) needle and optimal sonic plane. This position represents the target position of the manipulator and the manipulator is also preferably controlled in such a way that this target position is adjusted (changed) when the (biopsy) needle or the instrument is moved. This means that the control device is preferably configured such that it moves the manipulator with the medical visualization device in such a way that the medical visualization device follows (tracks) a movement of the instrument.

An additional marker is preferably assigned to the medical visualization device in order that the location of the medical visualization device can be detected, and the control device is additionally configured to determine the location of the medical visualization device with the aid of the additional marker. The location of the visualization device is known per se because the arrangement of the device on the manipulator is known and thus the spatial coordinates of the device can be determined at any time on the basis of the manipulator position. Sensors are also known, by means of which the position of the marker in space, and thus relative to the sensor, can be determined in a very precise manner. However, an additional marker helps to determine the relative spatial arrangement of the visualization device and the instrument relative to one another, and in particular when the location of the manipulator and/or of the sensor, with which the marker is detected, is not fixed relative to one another. In such cases, the use of two markers, i.e., on the visualization device and on the instrument, permits the determination of the relative location of the two markers (and thus of the device and the instrument) relative to one another. This is in particular the case when both have the same type of marker, which markers are detected by the same sensors. The system detects for example the markers and supplies the origin of the marker coordinate systems to the control device. Said control device can then perform the necessary transformation calculations.

Particularly preferably, the markers are optical markers, and a sensor in the form of a camera device is assigned to the control device, which is configured to detect the optical markers and their location in space. For example, the markers can be infrared light-reflecting spheres, and the camera device can be a stereo camera. With the aid of the stereo camera it is possible to determine the position and orientation in space of the instrument and, if appropriate, of the visualization device if it too has a corresponding optical marker, by which means the location can be calculated.

The manipulator is preferably a multiaxial articulated robot, the axles of which are provided with sensors for detecting the forces and/or torques acting on the axles. With the aid of the sensors it is possible to define force limits for the manipulator which it cannot exceed, for example when it presses the visualization device against the body of a patient. In this regard it is particularly preferred that the control device is configured to control the robot or articulated robot in such a way that the medical visualization device is pressed against the patient's body with a defined force. The defined force is preferably a range in order to ensure that the device is guided against the patient's body with sufficient force, but that determined maximum forces are not exceeded.

It is generally preferable that the medical visualization device comprises or is an ultrasonic probe. It is also generally preferable that the surgical instrument comprises or is a needle and in particular a biopsy needle.

The present invention furthermore relates to a method for robot-assisted medical treatment of a patient, comprising the following steps:
determining the location of a medical visualization device, which is mounted on a manipulator, in particular a multiaxial articulated robot, in order for it to be moved by said manipulator;
determining the location of a medical instrument relative to the location of the medical visualization device;
moving the manipulator with the medical visualization device depending on the relative location of the medical instrument and the medical visualization device.

The above information, technical explanations, examples and advantages, which was provided in connection with the system, all likewise apply in an unrestricted manner to the method. The visualization device thus comprises or is for example preferably an ultrasonic probe and the medical instrument comprises or is a (biopsy) needle, a catheter, a radiation source, etc.

The method preferably also comprises the movement of the manipulator, depending on the relative location of medical instrument and medical visualization device, in such a way that the medical visualization device detects at least a part of the instrument and follows a movement of this part of the instrument. The visualization device or the manipulator thus “tracks” the instrument. It is not absolutely necessary that the full instrument is detected by the image plane of the device; in practice it is usually sufficient that the important parts of the instrument, such as the tip of a needle, are detected by the visualization device and are preferably tracked.

The method preferably also comprises:
defining a target point in space, and
automatic movement of the manipulator when the medical instrument nears the target point such that the medical visualization device is orientated so as to detect the target point in space. A target point can for example be a certain point in the patient's body, such as lymph nodes or a tumor, or the like, which is to be treated. This target point is detected (defined) and recorded for example in the control device of the manipulator, so that the manipulator can orientate the visualization device at any time on command such that the target point is detected, i.e. depicted or visualized. This can be advantageous in certain procedures on a patient because in the case of sufficient proximity of the instrument to the desired target point for example, a focusing of the visualization device on this target point is more helpful for the surgeon than a focusing (orientation) on a part of the instrument.

The present system and the method provide the advantage that the surgeon is relieved of the task of orientation and alignment of the visualization device, as this task is assumed by the control device and the manipulator. As a result, the surgeon or physician is able to concentrate on his actual task, for example, the puncturing of a structure of interest. The invention permits a quality enhancement of navigated, image-based biopsies through the use of a manipulator, which holds the visualization device and moves it in such a way that the information of interest is always on the screen.

### 4. EXEMPLARY EMBODIMENT

The present invention is described in greater detail below with reference to the attached figures, in which:

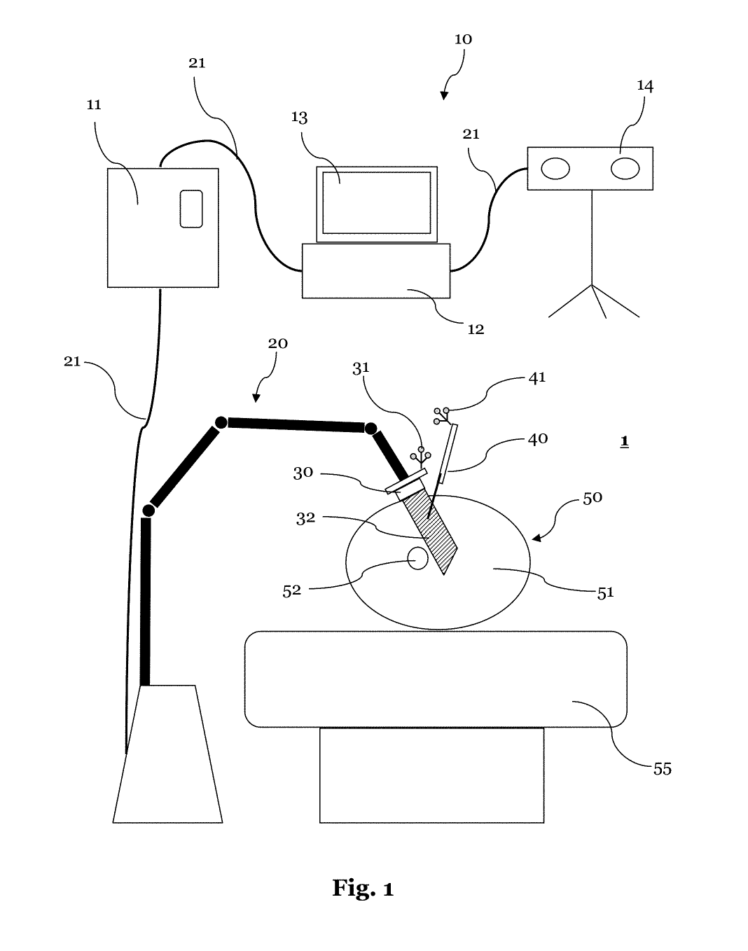

FIG. 1 shows, in a schematic depiction, a system according to the invention for robot-assisted treatment of a patient; and

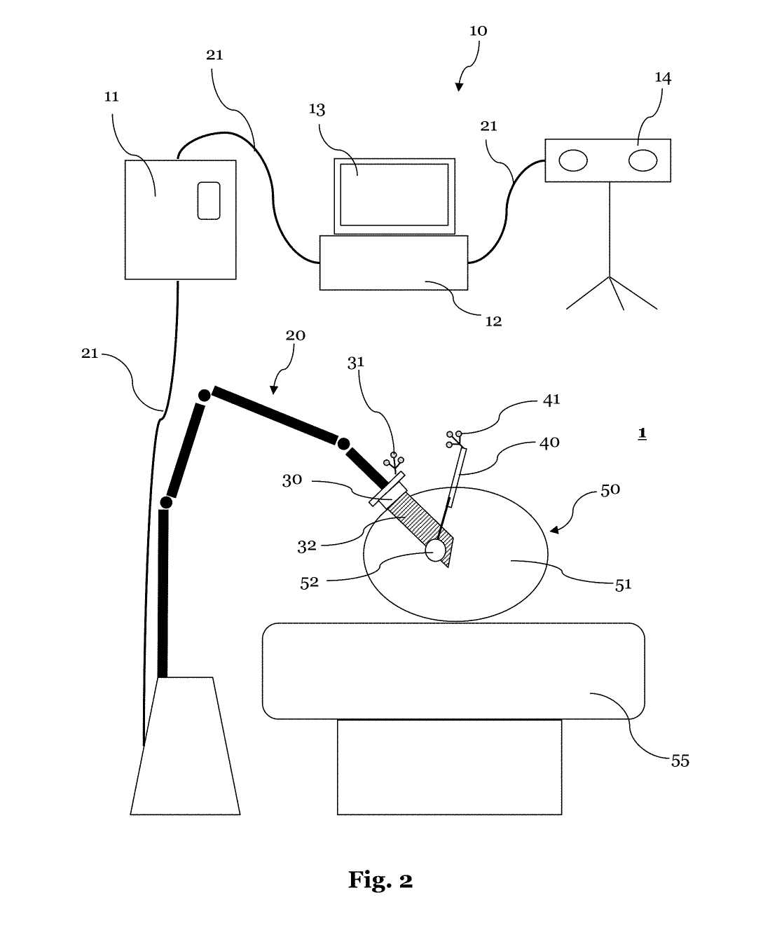

FIG. 2 shows the system of FIG. 1 with the manipulator and the visualization device in another position.

FIGS. 1 and 2 show, in a schematic and exemplary manner, a system 1 according to the invention for robot-assisted treatment of a patient 50. The system comprises a control device 10, which has a robot controller 11, a computer 12 and a stereo camera 14. The patient 50 lies on an operating table 55 and in the depiction 51 serves to indicate a sectional view through the throat of the patient 50. A target point 52 to be examined or treated, such as a tumor or the like, is situated in the throat 51. The treatment is to be realized by means of a surgical instrument 40, in particular a biopsy needle 40, which is manually guided by a surgeon in the depicted example. Alternatively, the biopsy needle 40 could also be guided by an additional manipulator. The biopsy needle 40 is to be guided to the target point 52. In order to make the guiding of the biopsy needle 40 easier for the surgeon, or to make said guiding possible at all, a medical visualization device 30 in the form of an ultrasonic probe 30 is used (preferably in conjunction with a computer/a processing unit and an HMI or monitor, by means of which the detected (image) data of the medical visualization device 30 is actually conveyed).

The robot controller 11 serves to control a multiaxial articulated robot 20 (or manipulator 20). The controller 11 and the articulated robot 20 are in communication with one another via data lines 21. Additional data lines 21 serve for communication with the additional components of the control device 10. The articulated robot 20 supports and moves the ultrasonic probe 3o. The ultrasonic probe 30 is pressed by the articulated robot 20 against the body of the patient 50 in order to produce ultrasonic images of the inside of the patient's body. The ultrasonic images are transferred via the data lines 21, processed in the computer 12 and then displayed on the monitor 13. The reference numeral 32 indicates the image plane (sonic plane) of the ultrasonic probe 30. The image plane or sonic plane of the probe is usually only a few millimeters thick, which means that the probe must be orientated very precisely in order to provide informative images.

The orientation of the probe and the pressing of the probe are realized by means of the manipulator or articulated robot 20, which means that a surgeon is relieved of these tasks. For this purpose, it is advantageous that the robot or articulated robot 20 is provided with force sensors and operates with force regulation, so that it presses the ultrasonic probe 30 with a defined force onto the skin surface of the patient 50. To do this, the robot controller 11 calculates the path to the target position and target orientation using the ancillary conditions “retain skin contact with defined force”, “no collision with ultrasound needle”, “no collision with marker”, etc.

In the exemplary embodiment, the biopsy needle 40 is provided with an optical marker 41. The stereo camera 14 of the control device 10 detects the marker 41 and supplies the origin of the marker coordinate system to the robot controller 11 or to the computer 12 in order to determine the location of the biopsy needle 40. The robot controller 11 then calculates the optimal location of the ultrasonic probe 30 (target position and target orientation) depending on the location of the biopsy needle 40. Because the location of the ultrasonic probe 30 is known on the basis of the current (articulated) robot position or manipulator position or can be calculated therefrom, and the extension and the orientation of the sonic plane 32 is also known, it is thus possible to orientate the probe 30 automatically. In FIG. 1, the probe 30 is directed towards the tip of the biopsy needle 40 and the needle tip (or biopsy needle tip) is detected by means of the sonic plane 32. The surgeon can follow on the monitor 13 the movement of the needle tip through the body of the patient 50 and guide the biopsy needle 40 in a correspondingly targeted manner to the target point 52.

In FIG. 2, the biopsy needle 40 punctures the target point 52 in order to take a tissue sample for example at this location. The manipulator 20 has relocated the probe 30 accordingly, so that the sonic plane 32 is still directed towards the needle tip and detects said needle tip such that the position of the biopsy needle 40 can be depicted on the screen 13. This relocation is realized automatically by the robot controller 11 on the basis of the changed location of the biopsy needle 40. The stereo camera 14 detects the marker 41 and thus the changed location of the biopsy needle 40, so that the control device 10 initiates the corresponding movements of the articulated robot 20.

In the depicted example, the ultrasonic probe 30 is also provided with an additional marker 31, which advantageously functions according to the same principle as the marker 41. The additional marker 31 can simplify the determination of the relative spatial location of the biopsy needle 40 and the probe 30 relative to one another.

The update rate of the system is preferably similar to the update rate of the tracking system (for example, 30-90 Hz or preferably 40 to 80 Hz), so that the articulated robot or manipulator can maintain the depiction of the biopsy needle 40 in the ultrasonic plane during the entire procedure. The articulated robot thus follows even the smallest movements of the biopsy needle 40, i.e., the biopsy needle 40 is tracked by the articulated robot and thus by the ultrasonic probe. The high update rate has the advantage that only small movements of the articulated robot are to be expected, as significant movements must be prevented for safety reasons.

### LIST OF REFERENCE NUMERALS

1 system
10 control device
11 robot controller
12 computer
13 screen
14 stereo camera
20 robot
21 data line
30 ultrasonic probe
31 marker
32 sonic plane
40 biopsy needle
41 marker
50 patient
51 cross section through throat
52 target point
53 operating table

1. A system for robot-assisted medical treatment of a patient; comprising:
a manipulator, in particular a multiaxial articulated robot,
a medical visualization device, which is mounted on the manipulator in order to be moved by said manipulator;
a medical instrument, which is provided with at least one marker in order that the location of the medical instrument can be detected; and
a control device, which is configured to determine the location of the medical instrument with the aid of the marker and to move the manipulator with the medical visualization device depending on the determined location of the medical instrument.

2. The system according to claim, wherein the control device is configured to move the manipulator with the medical visualization device depending on the location of the medical instrument in such a way that the medical visualization device detects at least a part of the instrument.

3. The system according to claim 2, wherein the control device is configured to move the manipulator with the medical visualization device in such a way that the medical visualization device tracks a movement of the instrument.

4. The system according to claim 1, wherein an additional marker is assigned to the medical visualization device in order that the location of the medical visualization device can be detected and the control device is also configured to determine the location of the medical visualization device with the aid of the additional marker.

5. The system according to claim 1, wherein the manipulator is a multiaxial articulated robot, and wherein the axles of the articulated robot are provided with sensors for detecting the forces and/or torques acting on the axles.

6. The system according to claim 5, wherein the control device is configured to control the articulated robot in such a way that the medical visualization device is pressed with a defined force against the body of the patient.

7. The system according to claim 1, wherein the markers are optical markers, and a camera device is also assigned to the control device, which is configured to detect the optical markers and their location in space.

8. The system according to claim 1, wherein the medical visualization device is an ultrasonic probe.

9. The system according to claim 1, wherein the surgical instrument is a biopsy needle.

10. A method for robot-assisted medical treatment of a patient; comprising the following steps:
determining the location of a medical visualization device, which is mounted on a manipulator, in particular a multiaxial articulated robot, in order for it to be moved by said manipulator;
determining the location of a medical instrument relative to the location of the medical visualization device;
moving the manipulator with the medical visualization device depending on the relative location of the medical instrument and the medical visualization device.

11. The method according to claim, wherein the movement of the manipulator is realized depending on the relative location of the medical instrument and the medical visualization device in such a way that the medical visualization device detects at least a part of the instrument and follows a movement of this part of the instrument.

12. The method according to claim 10, additionally comprising:
defining a target point in space, and
automatic movement of the manipulator when the medical instrument nears the target point, so that the medical visualization device is orientated so as to detect the target point in space.

13. The method according to claim 11, additionally comprising:
defining a target point in space, and
automatic movement of the manipulator when the medical instrument nears the target point, so that the medical visualization device is orientated so as to detect the target point in space.

14. The system according to claim 2, wherein an additional marker is assigned to the medical visualization device in order that the location of the medical visualization device can be detected and the control device is also configured to determine the location of the medical visualization device with the aid of the additional marker.

15. The system according to claim 2, wherein the manipulator is a multiaxial articulated robot, and wherein the axles of the articulated robot are provided with sensors for detecting the forces and/or torques acting on the axles.

16. The system according to claim 15, wherein the control device is configured to control the articulated robot in such a way that the medical visualization device is pressed with a defined force against the body of the patient.

17. The system according to claim 2, wherein the markers are optical markers, and a camera device is also assigned to the control device, which is configured to detect the optical markers and their location in space.

18. The system according to claim 2, wherein the medical visualization device (30) is an ultrasonic probe.

19. The system according to claim 2, wherein the surgical instrument is a biopsy needle.
